(19) United States
(12) Patent Application Publication
(10) Pub. No.: US 2014/0186456 A1
(43) Pub. Date: Jul. 3, 2014

(54) Tripartite Bioactive Composition Providing Anti-Oxidant and Anti-Inflammatory Effects with Immune System and Stem Cell Production Enhancement

(71) Applicants: Lillian M. Day, Henderson, NV (US); Tony A. Sampson, Henderson, NV (US)

(72) Inventors: Lillian M. Day, Henderson, NV (US); Tony A. Sampson, Henderson, NV (US)

(21) Appl. No.: 14/240,018

(22) PCT Filed: Aug. 25, 2012
(86) PCT No.: PCT/US12/52411

§ 371 Date: Feb. 20, 2014

Related U.S. Application Data
(60) Provisional application No. 61/527,605, filed on Aug. 25, 2011.

(51) Int. Cl. A61K 35/20; A61K 31/05; A61K 31/737

(52) U.S. Cl. CPC A61K 35/20; A61K 31/737; A61K 31/05
USPC 424/535

(57) ABSTRACT

The inventive tripartite composition is a combination of fucoidan, trans-resveratrol, and bovine colostrum in effective quantities, in the range of from 1:1.5:1.5 to 1:10:10 in weight percent. This composition provides antioxidant protection of cells against free radicals, strengthens the immune system, and enhances cellular function and stem cell population, thereby providing renewal and repair throughout the body. The inventive composition can be administered by capsules, but is preferably applied in liquid form to the oral cavity, such as by sublingual dropper or by intraoral spray. This sublingual, intraoral application method of administration provides maximum absorption, thus minimizing cost, avoiding inactive ingredients that are present in tablets and capsules, increasing consumer convenience, acid eliminating stomach irritation.

### CROSS-REFERENCE TO RELATED APPLICATIONS

This application claims priority to International Application No PCT/US12/52411, filed 25 Aug. 2012, which claims priority to U.S. Provisional Application No. 61/527,605, filed 25 Aug. 2011.

### TECHNICAL FIELD

The present invention relates generally to the field of bioactive compositions, and more particularly to a tripartite bioactive composition from natural sources having anti-oxidant, anti-inflammatory, and cell-renewing properties.

### BACKGROUND ART

To improve health, medical practitioners may recommend antioxidant foods or supplements to decrease free radicals; anti-inflammatory foods, drugs and/or supplements to ease pain and calm allergic reactions; and supplements to improve the body's immune system response. Additionally, they acknowledge that the number of healthy adult stem cells circulating in the bloodstream is one of the greatest indicators of human health. However, providing the combination of these valuable benefits (anti-oxidant properties, anti-inflammatory effects, immune system enhancement, and increase in stem cell population) in an effective delivery method has proven elusive.

Adult stem cells (those that reside in the body from birth) are cells capable of asymmetric division, proliferation (self-renewal while maintaining the undifferentiated state) and potency (the ability of cells to differentiate into diverse specialized cell types), e.g., adult stem cells have the ability to multiply by cell division, differentiate and replenish other cells and regenerate damaged tissues. Once mobilized, stem cells migrate to areas of injury and inflammation to assist in replacement and repair.

Scientific research shows that as the body ages stem cell numbers and stem cell release rates from the bone marrow drop rapidly. A child has a very high percentage of stem cells in circulation in the blood stream. By the age of 65 years, the release rate has dropped by 90% (leaving only 10% of active stem cells circulating in the blood stream to fight disease and aging, compared to that of a child). Furthermore, mesenchymal stem cells that reside in the bone marrow and have a specific role in repair have declined rapidly. Cellese Regenerative Therapeutics reports that the ratio for a newborn is 1:10,000 mesenchymal stem cells (proportion of mesenchymal stem cells to total cells in the bone marrow). In comparison, a 30-year-old person has a proportion of 1:250,000 mesenchymal stem cells to total bone marrow cells and an 80-year-old person's ratio has dropped to only 1:2,000,000. Thus, a child can recover quickly due to an optimum level of stem cells that can rapidly differentiate as needed to meet the challenges of injury or disease. However, as the body ages, fewer and fewer stem cells are available. This leads to health problems, disease, a weaker immune system, aging, slower recovery from injury, and failure of the body to repair the diseased organs.

Additionally, as the body ages, the immune system—which protects against disease by identifying and killing pathogens and tumor cells also weakens. The body is left with fewer defenses against aging, pathogen challenges, and disease.

Dr. Burton Feinerman leading physician in the study of aging, previously Chief of Medicine at the U.S. Army 98th General Hospital in Germany and Chairman of Medicine at Miami General Hospital, currently President and CEO of StemCellRegenMed) states the problem as follows: “On the whole, however, it is apparent that the number of stem cells in an organ and their functioning change as the body ages. From the point of therapeutics there are two approaches to treat stem cell aging in organs: (a) stimulate existing stem cells in various organs to increase in numbers, or (b) administer specific lines of stem cells to repair damaged tissue and introduce new young healthy cells to the organ.” Many effective treatments to increase stem cell numbers employ costly and invasive techniques (such as the introduction of autologous bone marrow stem cells, autologous adipose stem cells, allogeneic human umbilical cord blood stem cells, or full stem cell replacement therapy). However, a natural substance, fucoidan, which is a derivative of brown seaweed, is also known to encourage stem cell production.

Known anti-inflammatory supplements include resveratrol (3,5,4′-trihydroxy-trans-stilbene), a stilbenoid and a phytoalexim naturally produced by several plants when under stress or attack by pathogens, such as bacteria or fungi.

Bovine colostrum from pasture-fed cows contains immunoglobulins specific to many human pathogens, such as Escherichia coli, Cryptosporidium parvum, Salmonella, Staphylococcus, and rotavirus. It is a natural source of growth and immune factors including immunoglobulins, and, consequently, beneficial to the human body. Though a number of bovine colostrum powders of varying grades are commercially available, a high percentage of conventional colostrum is not of high quality, and the varying grades of colostrum are not equally effective or beneficial. Bovine colostrum is known to improve immune system function. An example of use of bovine colostrum (with other ingredients) in a lotion for application to the skin is disclosed in PCT Publication No. WO2007/000651 by Rosa Gobbi.

Thus, though various techniques and products have been developed to address a single problem, the need remains for a product that provides an effective combination of anti-oxidant properties, anti-inflammatory effects, immune system enhancement, and stem cell population increase.

### SUMMARY OF INVENTION

The present invention relates to a tripartite bioactive composition comprising a combination of bovine colostrum, trans-resveratrol, and fucoidan in effective quantities; also disclosed is an efficient intraoral means of administration. The inventive composition stimulates an increase in endothelial, endogenous, hematopoietic and mesenchymal stem cell numbers, strengthens the immune system, and provides antioxidant protection of cells, tissues and the entire organism against free radicals. Cellular function is enhanced leading to renewal and repair throughout the body and leading to an increased ability to identify and kill pathogens and tumor cells.

The synergistic effect of the three high grade ingredients, all utilizing specific pathways, optimizes the effectiveness of this inventive formulation.

The activation of adult stem cells is done through a combination of the following three active principals:

(1.) a high quality bovine colostrum providing an abundant amount of vital growth factors (IGF-1, IGF-2, transforming GFs A and B, epidermal-GE, fibroblast-GF and platelet-derived GF plus many immune factors including immunoglobulins, peptides, cytokines);

(2.) A high purity trans-resveratrol that supports the enrichment and proliferation of mesenchymal stein cells that reside in the bone marrow. (For example, the purity can be in the range of 95-100% pure.); and

(3.) An highly purity fucoidan that increases the expression of CD34+ cells. (For example, the purity can be in the range of 80-100% pure.)

The preferred method of administration of the inventive nutritive composition is through intraoral spray application though other delivery methods, such as by capsules, tablets, transdermal patches, liquids, and the like, are within the scope of the invention. The preferred intraoral spray administration allows the inventive composition to be absorbed more efficiently; therefore, an effective amount of the inventive composition can be delivered for a much lower cost.

An object of the present invention is to provide a tripartite bioactive composition that increases endothelial, endogenous, hematopoietic and mesenchymal stem cell numbers.

An additional object of the present invention is to provide a tripartite bioactive composition that is derived from natural sources.

Another object of the present invention is to provide a tripartite bioactive composition that has antioxidant properties.

A further object of the present invention is to provide a tripartite bioactive composition that has anti-inflammatory properties.

Another object of the present invention is to provide a tripartite bioactive composition that enhances and strengthens the body's immune system activity.

An additional object of the present invention is to provide a tripartite bioactive composition that can be administered in an efficient and cost-effective manner.

These and other objects and advantages of the invention will become more fully apparent from the description and claims which follow, or may be learned by the practice of the invention.

### BRIEF DESCRIPTION OF THE DRAWINGS

The preferred embodiments of the invention will hereinafter be described in conjunction with the appended drawings, provided to illustrate and not to limit the invention, where like designations denote like elements, and in which:

FIG. 1 is a graph showing a comparison of the granulocyte colony stimulating factor (GCSF) production of a composition of bovine colostrum, fucoidan, and resveratrol compared to a blue-green algae stem cell enhancing formula and compared to colostrum alone (provided by Chief Scientist, Dr. Gill Webster of Immuno Strategy, Auckland, New Zealand);

FIG. 2 is a graph showing a comparison of the interleukin 6 (IL-6) production of a composition of bovine colostrum, fucoidan, and resveratrol compared to a blue-green algae stem cell enhancing formula and compared to colostrum alone (provided by Dr. Gill Webster); and

FIG. 3 is a graph showing the total increased activity percentage of a composition of bovine colostrum, fucoidan, and resveratrol compared to a blue-green algae stem cell enhancing formula (provided by Dr. Gill Webster).

### DETAILED DESCRIPTION OF THE INVENTION

The present invention is directed toward a tripartite bioactive composition in an effective formulation for strengthening the immune system, enhancing cellular function, and increasing stem cell population, in which the main active components are high quality bovine colostrum, trans-resveratrol, and fucoidan. In addition, an advantageous intraoral spray method for administering the inventive composition is presented that allows maximum absorption, thereby minimizing cost, as the quantity of the active components required to achieve the desired blood stream concentration is reduced compared to other administration methods.

The active components of the inventive composition are derived from natural sources. The inventive composition has been shown to be safe to use, without noted side effects. The intraoral spray administration method is not only effective, but also avoids irritation of the stomach and avoids the nutrients being exposed to the acid and bile of the GI tract, so they remain bioactive.

The active components, fucoidan, trans-resveratrol, and colostrum, are combined in liquid form suitable for spray application. As an example which supports but does not limit the essence of the present invention, the percentage of fucoidan is from 10-25% by weight, the percentage of trans-resveratrol is from 25-60% by weight, and the percentage of bovine colostrum is 25-60% by weight. As an example which supports but does not limit the essence of the present invention, the active components, fucoidan, trans-resveratrol, and bovine colostrum are preferably in the proportion, by weight, of approximately 1:2.5:2.5. Thus a commercial preparation for retail sale might contain 6 mg fucoidan (containing over 80% cell components, and preferably over 85% cell wall components), 15 mg trans-resveratrol (as a Polygonal cuspidate extract of approximately 98% resveratrol), and 15 mg bovine colostrum (approximately igG 30%) per dose with 2 doses recommended per day. Small quantities of acceptable pharmacologic additives may also be included. Preferably, all three components are of the highest grade available and are sourced from countries that are known to produce the world's highest quality and most refined components.

### Colostrum

The colostrum used in the tripartite bioactive composition is bovine colostrum. Bovine colostrum is non-species specific. It contains the immune and growth factors found in other mammalian species sources; thus, it can significantly benefit all mammals, including humans. Calves are born without immunity to most disease-causing organisms; therefore, their mothers' colostrum must contain a very large amount of immune and health factors. As a result, bovine colostrum has approximately 20 times the growth and immune factors of human colostrum, making it a superior source of colostrum.

Prior to the development of antibiotics, colostrum was the main source of immunoglobulins used to fight infections, The first oral vaccine against polio was developed using immunoglobulins from bovine colostrum. In response to the recent unfortunate development of antibiotic-resistant strains of pathogens, there has been a significant return to research focusing on this proven, natural alternative to antibiotics.

Many documented clinical studies show the effects of colostrum on athletic endurance and performance. Also, colostrum is safe to use; it is one of the few natural supplements that is cleared by the international Olympic Drug Committee for use by Olympic athletes. Colostrum is well-known in the athletic community as a means to increase lean body mass without the dangerous side effects found in steroids. It also helps keep the athlete healthy by accelerating the healing of athletic injuries and boosting the immune system, which may become depressed after extreme exercise leaving the athlete susceptible to infectious diseases. Bovine colostrum has been scientifically proven to help reduce leaky gut syndrome that is a common condition for those who work in warm climates, in particular athletes and military personnel. Compared to whey, colostrum supplementation produces a significant increase in lean body mass, anaerobic power, performance time, and sprint performance. Marathon runners, who experience a significantly higher incidence of upper respiratory tract infections (URI) in the weeks leading up to and following a marathon, had a much lower rate of URI when receiving bovine colostrum supplementation as compared to a placebo.

Preferably, high quality bovine colostrum comprising more than 30% IgG is used in the inventive composition. High quality bovine colostrum is a rich source of growth factors (epithelial growth factor (EgF), insulin-like growth factor-I and II (IGF-1 and IGF-II), fibroblast growth factor (FgF), platelet-derived growth factor (PDGF), transforming growth factors A & B (TgA and B), growth hormone (GH).

IGF-1 is a hormone similar in molecular structure to insulin. It has been shown to enhance lean muscle development and athletic performance. IGF-1 when taken in its natural form is not banned by the International Olympic Committee, whereas synthetic forms are. Doctor J. O. Serrentino (who has a Ph.D. in environmental medicine and is a veterinarian, researcher, and consultant to biopharmaceutical companies) has reported in The Cytokine Handbook that growth factors stimulate, mobilize and cause cellular differentiation and proliferation, plus influence repair and healing at the cellular level.

Preferably, fresh bovine colostrum that is processed without freezing and is spray-dried only for storage (ensuring the composition, molecular structure, and nutrients are preserved) is used in the inventive tripartite bioactive composition. For example, New Zealand bovine colostrum has gained world-wide respect and recognition for its purity and quality. Additionally, the intensely managed, year-round pasturelands in New Zealand result in grass-fed cattle, requiring little or no supplemental grains or soy to be fed. Furthermore, New Zealand cattle calve at the same time of year, allowing the colostrum to be collected fresh and processed immediately. This compares favorably to colostrum from other locales that generally require freezing prior to processing; the re-thawing and heat treatment involved in this process destroys vital nutrients. Colostrum that is processed fresh is far more effective and pure (up to 90% more wholesome and nutritive) than frozen processed colostrum. The spray drying method fully preserves the nutrients and goodness of the fresh colostrum.

The use of bovine colostrum also provides other advantages. Bovine colostrum protects the digestive tract from infection; the immunoglobulin A (IgA) within the colostrum protects the mucosal lining.

Bovine colostrum has anti-inflammatory properties. Bovine colostrum has been shown to be beneficial in treating chronic inflammatory diseases, such as various forms of arthritis and Crohn's disease, which have been linked to inflammation of the human gastrointestinal tract. Inflammation is reduced due to the tumor necrosis factor-alpha (TNF-α) receptors in colostrum that bind TNF-α, a cytokine which controls the inflammatory cytokine cascade. Also, bovine colostrum contains interleukin-1ra (IL-1ra), the IL-1 receptor protein, which also acts to inactivate the inflammatory activity of IL-1.

The lactoferrin within the bovine colostrum also inhibits the production of inflammatory cytokines. Lactoferrin has strong antioxidant characteristics which take place by two separate mechanisms: binding catalytic iron generated during cell destruction and binding bacterial lipopolysaccharide (LPS), which causes a decrease in LPS bioactivity and an increase neutrophil O₂ production.

The proline rich peptides (PRP) within the bovine colostrum also act as an oxidative stress regulator.

### Trans-Resveratrol

The second main active component of the inventive tripartite bioactive composition is trans-resveratrol. Preferably full-spectrum trans-resveratrol of highest quality (such as a 98%-100% trans-resveratrol) is used. The trans-resveratrol used in the inventive tripartite composition is most preferably an extract derived from Polygonum cuspidatum, Japanese knotweed. For example, a recommended dosage of trans-resveratrol to be delivered via the tripartite bioactive composition is between 10 mg and 50 mg per day. It has been discovered more recently that relatively small dosages of trans-resveratrol can be very effective. Results from a study were published in the 2010 Journal of Endocrinology and Metabolism. Within this study, Husam Ghanim, PhD of the University of Buffalo made three important statements as to why this study was significant: 1) the test subjects were humans; 2) the study used only 40 milligrams of resveratrol (one dosage a day for 6 weeks); 3) the study used a Japanese Knotweed extract, showing the effectiveness of resveratrol derived from that source.

Resveratrol is known for a long list of health benefits. Resveratrol may enhance health and support longevity via several mechanisms. Studies show that trans-resveratrol may be a useful supplement for healthy adults wishing to reduce their risk of chronic disease and live a long and healthy life. Studies to date suggest that resveratrol is safe and nontoxic.

Resveratrol supports cardiovascular health and protects blood-vessel cells. It also provides anti-oxidant protection, boosts cellular energy production, balances the immune system, provides anti-inflammatory benefits, prevents joint disease, improves athletic endurance, lowers blood sugar counts, and possibly prevents or eliminates certain types of cancer. Research by the Harvard Medical School has found that trans-resveratrol increases the production of a protein called SIRT1, which increases the life span in several animal species and may increase human lifespan. This finding was announced in 2003 when Doctors Baur and Sinclair of Harvard Medical School discovered that resveratrol increased the lifespan of organisms and animal species by 20-40%. In 2009, Christopher Paul Erdman found that resveratrol enriched adipose-derived mesenchymal stem cells. Many published medical studies have continued since this time showing very favorable results.

Additionally, by increasing blood flow, resveratrol may protect not only the heart and arteries, but also the brain. One study indicating this is titled “Effects of Resveratrol on Cerebral Blood Flow Variables and Cognitive Performance in Humans: A Double-Blind, Placebo-Controlled, Crossover investigation,” published in American Journal of Clinical Nutrition, June 2010 91: 6 1590-1597. The authors of the study concluded that “Resveratrol administration resulted in dose-dependent increases in cerebral blood flow during task performance, as indexed by total concentrations of hemoglobin, There was also an increase in deoxyhemoglobin after both doses of resveratrol, which suggested enhanced oxygen extraction, that became apparent toward the end of the 45-min absorption phase and was sustained throughout task performance.”

Another recent article from the same journal (American Journal of Clinical Nutrition July 2010 92: 1 5-15) suggests that resveratrol may be helpful in treating obesity. The article, entitled “Resveratrol Regulates Human Adipocyte Number and Function in a SIRT1-Dependent Manner”, states that the “data suggest that resveratrol influences adipose tissue mass and function in a way that may positively interfere with the development of obesity-related comorbidities.” Furthermore, the study by Doctors Sinclair and Baur in 2003 showed resveratrol works by entering a cell and activating the enzyme called SIRT1. This enzyme stimulates the growth of new mitochondria (the engines of cells) that increase the metabolic rate, that was found to help slow the aging process, Furthermore, the September 2010 issue of Clinical Endocrinology and Metabolism reported resveratrol intake reduced inflammatory and oxidative stress in healthy adults.

Recent studies in the United Kingdom suggest that resveratrol may help prevent or reduce the risk of Alzheimer's disease. The study titled “Therapeutic Potential of Resveratrol in Alzheimer's Disease” published in BMC Neuroscience 2008, 9 (Suppl. 2) makes the following statement: “Several epidemiological studies indicate that moderate consumption of red wine is associated with a lower incidence of dementia and Alzheimer's disease, Red wine is enriched in antioxidant polyphenols with potential neuro-protective activities. Despite skepticism concerning the bioavailability of these polyphenols, in vivo data have clearly demonstrated the neuro-protective properties of the naturally occurring polyphenol resveratrol in rodent models for stress and diseases. Furthermore, recent work in cell cultures and animal models has shed light on the molecular mechanisms potentially involved in the beneficial effects of resveratrol intake against the neurodegenerative process in Alzheimer's disease (AD).”

Further, a study by the University of South Florida appearing in the American Journal of Physiology—Gastrointestinal and Liver Physiology, published by The American Physiological Society points to resveratrol as a possible treatment for alcoholic fatty liver disease and as a way to prevent the disease in those who are at risk, but have not developed it.

### Fucoidan

The third component of the inventive tripartite bioactive composition is fucoidan. Fucoidan is a complex carbohydrate (extract-sulphated polysaccharide), multi-functional nutrient from the sea. High quality Fucoidan is only obtained from special species of brown seaweed. This nutrient has been studied extensively, particularly for its health benefits, with growing evidence that supports its cancer fighting and weight loss abilities. Fucoidan is rich in calcium, iodine, zinc, iron, selenium and vitamin A; these nutrients are essential for proper functioning of the immune, circulatory and neurologic systems. Over 700 published papers identify benefits of fucoidan. Fucoidan has been shown to mobilize stem cells and defend against degeneration and improves immune ability. For example, the research of Japanese scientist, Dr. Kyosuke Owa, shows that fucoidan stimulates the production of immune cells, providing the body with a stronger defense against invading bodies like bacteria, viruses, fungi, parasites, and even cancer. Dr. Owa noted similarities to fucoidan polysaccharide and human breast milk and called fucoidan “the plant equivalent of mother's milk.”

Fucoidan has been shown to increase the beneficial activity of lymphocytes. Lymphocytes are white blood cells active in the immune system. They include T cells involved in cell-mediated immunity, B cells involved in humeral immunity, and natural killers that defend the body from tumors and infections. Lymphocytes move from the blood into secondary lymphoid tissues to perform their functions. The initial step in this process is the binding of lymphocytes to high endothelial venules (HEV), which fucoidan has been shown to increase. The Memorial Sloan Kettering Cancer Center found fucoidan acts to block cancer cells from developing and growing through a process called apoptosis. In 2006 a Pub Med study was released by the Society of Hematology and Stem Cells confirming oral fucoidan significantly amplified the CXCR4+ HPC (hematopoietic progenitor stem cell) population. The importance of increasing hematopoietic progenitor stem cells is exemplified by the fact that transplantation of hematopoietic progenitor stem cells (HPC) is an important treatment modality for a variety of neoplastic diseases.

The physiological and biochemical effects of fucoidan have been examined in several small-scale in vitro and animal studies. Fucoidan was reported to inhibit hyperplasia in rabbits. It has also been shown to induce apoptosis (cell death involving eliminating old and unhealthy cells) in isolated human lymphoma cell lines in vitro.

Fucoidan has also been shown to minimize the effects of photoaging. Destruction of collagen is a hallmark of photoaging. The major enzyme responsible for collagen digestion, matrix metalloproteinase-1 (MMP-1), is induced by exposure to sunlight including ultraviolet-B (UVB). The UV radiation-induced increased MMP-1 expression is mediated by extracellular signal-regulated kinase (ERK) activation. Research published in Biol. Pharm. Bull. 31(2) 284-289 (2008) entitled “Fucoidan Inhibits UVB-Induced MMP-1 Expression in Human Skin Fibroblasts” involved an investigation of the inhibitory effects of fucoidan MMP-1 expression by various in vitro experiments, and elucidated the pathways of inhibition. This study demonstrated the inhibitory effect of fucoidan MMP-1 expression via protein and mRNA assay. The researchers indicated that fucoidan may prevent UVB-induced MMP-1 expression by inhibiting the UVB-induced activation of the ERE, signaling pathway, Therefore, fucoidan is a potential agent for preventing and treating skin photoaging.

### Combination of Trans-Resveratrol, Bovine Colostrum and Fucoidan

Though a great number of health benefits are provided by the individual introduction into the human body of each of these three active principals, together the combination of trans-resveratrol, bovine colostrum and fucoidan work synergistically to boost cellular function by utilizing all the required pathways necessary. By stacking these three components together, the body receives a direct boost, allowing it to run more efficiently immediately and for a longer period of time. The prompt response is enabled by the quick uptake through the mucosal tissue and directly into the blood stream, bypassing the slower and less efficient stomach. The sustained beneficial response is due to the cooperation of these three active principals. The combination yields a greater effect with faster and longer lasting results.

The cooperation of the three principals provides a triple defense mechanism against pathogens and bacteria attempting to attack or enter the body. This is particularly important as the body ages and the immune system declines. Plus, the immune system is strengthened as quickly as possible (such as after surgery, a traumatic experience, injury or disease). As we age the effectiveness and absorption abilities of the gastrointestinal track also weaken therefore the way in which people take their health supplements becomes even more important.

The beneficial combination of trans-resveratrol, bovine colostrum and fucoidan in the quantities and quality described, compared to the equivalent single doses of the three principals, increases the following: activation of the body's adult stem cells, strengthening of the immune system, optimization of the anti-oxidant and anti-inflammatory effects, reinforcement of the ergogenic effect, and enhancement of the cellular renewal and repair.

Study participants taking the recommended dosage of the inventive tripartite bioactive composition report much improved energy levels, repair and recovery after surgery, illness or injury, increased concentration, and better sleep, waking more refreshed. They report these benefits taking effect within a matter of days. After one month of usage of the inventive tripartite composition, Doctor Kenneth Welker of Oregon Optimal Health wrote concerning his and his patients' response to the formulation. He stated, “We have received good comments regarding the use of this formulation in our office over the past few weeks. This includes increased energy, reduction of pain, and improvement of concentration. I recently started taking the formulation with a known rotator cuff tear of the left shoulder. It has been causing me pain every day for the past 6 months. Since starting the formula after one week, the pain was gone. I am now doing high intensity training without difficulty.”

One synergistic benefit occurs with regard to cell communication. Bovine colostrum contains protein molecules (cytokines) that are used extensively in intercellular communication. Each cytokine has a matching cell-surface receptor. The effect of a particular cytokine on a given cell largely depends on the presence and abundance of the complementary receptor on the cell surface. Unlike many polysaccharides, fucoidan does not compete for the same binding sites. Instead, when combined with resveratrol and bovine colostrum, fucoidan serves to enhance receptor activation. In addition, the trans-resveratrol targets the activation and enrichment of the mesenchymal stem cells. The stromal mesenchymal stem cells can differentiate into a variety of cell types. These stem cells help repair muscle, bone, cartilage and tendons. Both the stromal mesenchymal and hematopoietic stem cells work in concert to achieve tissue regeneration.

Beneficially, the combination of resveratrol, bovine colostrum and fucoidan activates the SIRT1 longevity gene. This specific combination evidences a synergistic effect that significantly modulates biomarkers of bone metabolism and inhibits pro-inflammatory enzymes such as COX-1 and COX-2. Chemopreventive agents, anticarcinogenic properties, cardio-protective effects and neuro-protective properties are significantly enhanced through this inventive composition as well.

The combination of the three components has been shown to provide powerful anti-oxidant and anti-inflammatory activity, strengthening of the immune system, enhancement of cellular function, increased energy, more restful sleep, healthy skin and nails, and an increase in stem cell population. These valuable benefits provide effective, continued renewal and repair throughout the body.

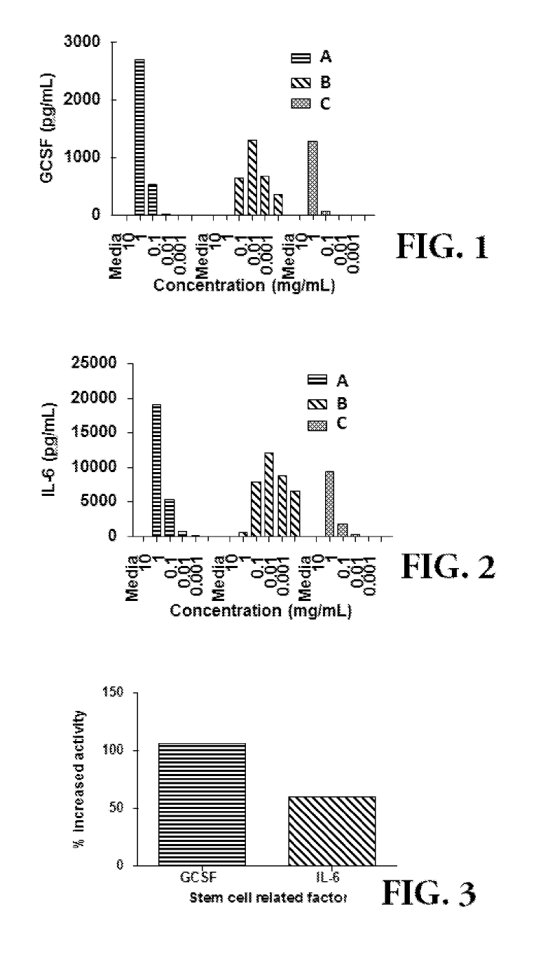

Independent scientific tests show that the combination of the three active principals (fucoidan, trans-resveratrol, and bovine colostrum) increase production of Granulocyte colony-stimulating factor (GCSF) and Interleukin 6 (IL-6) over the administration of one of the active principals alone. (GCSF stimulates the bone marrow to produce stem cells and to release them into the blood stream. IL-6 acts as both a pro-inflammatory and anti-inflammatory cytokine.) FIG. 1 shows a comparison of the GCSF production of a composition of bovine colostrum, fucoidan, and resveratrol (A) compared to a blue-green algae stem cell enhancing formula (B) and compared to colostrum (C) alone. Though this study was done with a lower purity composition of bovine colostrum, fucoidan, and resveratrol and with administration by capsules, as opposed to the preferred intraoral administration and higher grade components of the instant invention, the composition of bovine colostrum, fucoidan, and resveratrol in capsule form still shows a 100% increase in GCSF production over the blue-green algae formula, and also over colostrum alone. (The blue-green algae capsule used in this study has been marketed and sold as a stem cell enhancer since 2006.)

Similarly, FIG. 2 shows a significantly greater IL-6 production with the composition of bovine colostrum, fucoidan, and resveratrol compared to a blue-green algae stein cell enhancing formula and compared to colostrum alone. Further, FIG. 3 shows the total increased activity percentage of the composition of bovine colostrum, fucoidan, and resveratrol compared to a blue-green algae stem cell enhancing formula. Consequently, the combination of the three active principals of trans-resveratrol, bovine colostrum and fucoidan has been shown to have increased effectiveness compared to other formulations that purport to increase stem cell activity.

### Delivery Means

The combination of trans-resveratrol, bovine colostrum and fucoidan provides the benefits of anti-oxidant properties, anti-inflammatory effects, immune system enhancement, and stem cell population increase when administered by any delivery system. Therefore, the tripartite combination can be administered by any delivery means, including by tablet, capsule, cream, liquid, transdermal patch, sublingual (droppers), intramuscular, intravenous, or intraoral spray. The preferred delivery means are methods that bypass the digestive tract, such as transdermal patch, sublingual application of liquid with a dropper, and intraoral spray. These allow the full concentration of the inventive composition to enter the bloodstream at its highest potency level.

### Intraoral Administration

The preferred method of administration is by intraoral spray. The term “intraoral” is herein used to denote a sublingual or oral trans-mucosal application, as the inventive composition is applied in spray form to the oral cavity, specifically in the sublingual region of the oral cavity. The oral mucosa, via liposome transport, transfers the inventive composition, introduced by spray, into the sublingual region for absorption through the sub-mucosal tissue into the bloodstream. This delivery means provides more efficient absorption and, therefore, allows a smaller quantity to provide the full benefit of a larger quantity delivered by ingesting capsules, tablets, or liquids.

Intraoral (sublingual) spray has been shown to be an efficient delivery system. As stated by Ronald M. Lawrence, Ph.D. (a specialist in neurology, assistant clinical professor at UCLA School of Medicine, and former member of the National Advisory Council on Aging), “The intra-oral spray nutrient delivery system is the wave of the future. In my experience as a practicing physician, l find that spray delivery not only guarantees that the patient gets most of the nutrient delivered faster and more efficiently into the blood stream, but it also avoids gastrointestinal tract problems present with so many oral capsules, tablets, and liquids. The intra-oral spray method of delivery ensures that you get the full amount of the nutrient. This is especially important not only with older adults, but also many younger people who often fail to absorb the nutrients they ingest.”

The well-respected Physicians' Desk Reference, NPPDR #18, a commercially published compilation of manufacturers' prescribing information, verifies the efficacy of intraoral spray administration. It states the compared absorption rates as tablets 10%, capsules 20%, transdermal patches 45%, sublingual (dropper) 50%, intramuscular 90%, intraoral spray 95%, and intra-venous 100%. Thus the 95% absorption rate of intraoral spray provides an efficient delivery means for the inventive tripartite composition of the instant invention.

As the inventive composition is sprayed under the tongue instead of being ingested and digested, it bypasses the stomach by being transported through the sub-mucosa membrane and directly into the bloodstream. As a greater percentage of the bioactive composition is absorbed in this administration method, less of the inventive composition is required to reach an equivalent concentration in the blood. Thus, a lower dosage intake is required to achieve the desired bloodstream concentration as compared to other administration methods, resulting in a reduced cost.

The rapid uptake by the body allows the body's immune system to build up faster and respond more efficiently and effectively. Simultaneously, the levels of stem cells are enhanced more quickly, and the anti-oxidant and anti-inflammatory benefits begin immediately. Hence this leads to amore rapid response and feeling of wellbeing.

Further, the intraoral spray method of administration provides additional benefits. A portion of the population has difficulty in swallowing pills or capsules; the intraoral spray delivery meets the needs of this segment of the population. Many people have trouble with their digestive system, particularly as they age; this intraoral spray does not enter the gastrointestinal track, so it cannot irritate or upset the stomach. No water needs to be ingested when using the intraoral spray method of administration, as compared to pill and capsule administration. Tablets and capsules generally contain the added inactive ingredients magnesium stearate and silica which, though both are recognized as GRAS (Generally Regarded As Safe), may not be acceptable for people with certain medical conditions. Many capsules also contain emodin and fillers that are known to cause side effects.

The tripartite bioactive composition of the instant invention is preferably administered by periodic application, such as sublingual liquid drops or intra oral spraying. This periodic application delivers and maintains a uniform concentration of this composition in the blood, making therapeutic levels of the active principals available for absorption throughout the body.

Other advantages to intraoral sprays relate to convenience. The oral spray vial is small, slender, and compact, allowing the user to easily transport it in a pocket, briefcase or purse. Thus, it is convenient to use during a busy day or when traveling, particularly compared to carrying around a variety of bulky, noisy pill bottles.

### Preparation

The inventive composition is formulated in a way suitable for intraoral spray administration and prepared according to pharmaceutical methods. To prepare the inventive composition, the dry components are hydrated with purified water and are mixed gently, preferably by hand. A sufficient quantity of water is mixed with the three main components. This allows the pure, active compounds of the inventive composition to be extracted and dispensed as a liquid, such as for administration by sublingual or intraoral application, or to be formulated for transdermal patch administration. Extremely small quantities of other pharmacological ingredients may be used, such as natural vanilla flavor, citric acid and potassium sorbate. The liquid inventive composition is then preferably packaged in a spray vial for retail distribution. Optionally, the liquid inventive composition may be packaged in a small vial provided with a dropper for sublingual application

The frequency of application of the inventive composition to the sublingual area of the mouth will depend on the concentration of the formulation and the required effect, but, for example, could be 1 to 15 sprays a day. In an exemplary concentration, the spray may deliver 6 mg of fucoidan, 15 mg of bovine colostrum, and 15 mg of trans-resveratrol per 6 sprays. An exemplary standard daily dosage for adults is generally 12 sprays, which, therefore, delivers 12 mg of fucoidan, 30 g of bovine colostrum and 30 mg of trans-resveratrol per day.

Other means of delivery of the tripartite composition are also within the scope of the invention. For example, gel caps can be filled with the dry components for oral administration and absorption through the gastrointestinal tract. Or the components may be incorporated into a transdermal patch and delivered by absorption through the skin into the bloodstream.

In conclusion, the synergy of the three main components of the inventive bioactive composition supports a healthy immune system, provides anti-inflammatory effects, and increases the population of stem cells, This synergy produces improved health and enhanced repair and renewal within the body, while increasing the body's ability to ward off disease and pathogens. Though the composition of the instant invention may be delivered by any standard pharmaceutical delivery means, the preferred intraoral spray method of administration delivers an effective dose that enters the bloodstream at its highest potency level in non-irritating manner and at a reduced cost.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A bioactive composition, comprising as active principals fucoidan, trans-resveratrol, and colostrum.

2. The bioactive composition of claim 1, wherein said trans-resveratrol comprises an extract from Polygonum cuspidatum.

3. The bioactive composition of claim 1, wherein said trans-resveratrol comprises at least 98% trans-resveratrol.

4. The bioactive composition of claim 1, wherein said fucoidan comprises at least 85% cell wall components.

5. The bioactive composition of claim 1, wherein the ratio of said fucoidan, said trans-resveratrol, said colostrum is in the range of from 1:1.5:1.5 to 1:10:10 in weight percent.

6. The bioactive composition of claim 1, wherein said ratio of said fucoidan, said trans-resveratrol, said colostrum is in the proportion, by weight, of 1:2.5:2.5.

7. The bioactive composition of claim 1, wherein said colostrum comprises bovine colostrum.

8. The bioactive composition of claim 7, wherein said bovine colostrum comprises at least 30% IgG.

9. The bioactive composition of claim 7, wherein said bovine colostrum comprises a formulation prepared by spray drying.

10. The bioactive composition of claim 1, wherein said bioactive composition is administered in a dosage to provide between 10 mg and 50 mg trans-resveratrol and colostrum per day.

11. The bioactive composition of claim 1, wherein the percentage of said fucoidan is from 10-20% by weight, the percentage of trans-resveratrol is from 25-60% by weight, and the percentage of colostrum is 25-60% by weight.

12. The bioactive composition of claim 1, wherein said bioactive composition is provided in capsule form.

13. The bioactive composition of claim 1, wherein said bioactive composition is provided in liquid form in a container with a dropper, suitable for administration as liquid drops into the sublingual area of a human mouth.

14. The bioactive composition of claim 1, wherein said bioactive composition is provided in liquid form in a spray container, suitable for administration by intraoral spray to the sublingual area of a human mouth.

15. A method of treating a patient to increase stem cell numbers and provide anti-inflammatory effects, comprising administering a composition comprising a combination of fucoidan, trans-resveratrol, and bovine colostrum in quantities in the range of from 1:1.5:1.5 to 1:10:10 in weight percent by an intraoral spray delivery means.
